(19) United States
(12) Patent Application Publication
(10) Pub. No.: US 2016/0030533 A1
(43) Pub. Date: Feb. 4, 2016

(54) COMPOSITIONS AND METHODS OF TREATING FUNGAL AND BACTERIAL PATHOGENS

(71) Applicant: LOS ANGELES BIOMEDICAL RESEARCH INSTITUTE AT HARBOR-UCLA MEDICAL CENTER, Torrance, CA (US)

(72) Inventors: Michael R. YEAMAN, Redondo Beach, CA (US); Ashraf S. IBRAHIM, Irvine, CA (US); Scott G. FILLER, Rancho Palos Verdes, CA (US); John E. EDWARDS, JR., Palos Verdes Estates, CA (US)

(21) Appl. No.: 14/774,515

(22) PCT Filed: Mar. 14, 2014
(86) PCT No.: PCT/US14/28535

§ 371 Date: Sep. 10, 2015

Related U.S. Application Data
(60) Provisional application No. 61/787,380, filed on Mar. 15, 2013.

(51) Int. Cl. A61K 39/00; C07K 7/08; C07K 16/12; A61K 39/085; A61K 39/39; C07K 16/14; C07K 7/06; A61K 47/48

(52) U.S. Cl. CPC A61K 39/0002; C07K 7/06; C07K 7/08; A61K 47/4833; A61K 39/085; A61K 39/39; A61K 47/48284; C07K 16/14; C07K 16/1271; C07K 2319/00; A61K 2039/6081; A61K 2039/55505; A61K 2039/54; C07K 2317/76

(57) ABSTRACT

The invention features isolated polypeptides and conjugates including the amino acid sequence of any one of SEQ ID NOs: 3-11, or a variant sequence thereof having up to three substitutions, deletions, or additions to the amino acid sequence of any one of SEQ ID NOs: 3-11, wherein the polypeptide does not include more than 20 contiguous amino acids of SEQ ID NO: 2 or SEQ ID NO: 17. Additional polypeptides includes those of formula (I) [ZNZPVSSBSFSYT]ₙ and formula (II) [ZNUWOOBUFOYT]ₙ. The invention further features vaccines including such polypeptides or conjugates and methods of vaccination against candidiasis or a staphylococcal infection of both using same. In addition, the invention features antibodies that bind to the polypeptides or conjugates, and methods of passive immunization using same.

### FIELD OF THE INVENTION

In general, this invention relates to compositions and methods for providing host immune protection and passive immunoprotection against candidiasis and staphylococcal infections or both.

### BACKGROUND OF THE INVENTION

From the fungal kingdom, Candida species, a prevalent pathogen of healthcare-associated bloodstream infections, causes approximately 60,000 cases of hematogenously disseminated candidiasis per year in the United States, resulting in billions of dollars of healthcare expenditures. Despite current antifungal therapy, mortality remains unacceptably high. Because of the rising incidence of life-threatening candidiasis and high treatment failure rates, more effective prophylactic and therapeutic strategies are needed.

In the prokaryotic kingdom, Staphylococcus aureus is the leading cause of skin and skin structure infections including cellulitis and furunculosis, and is among the most common causes of bacteremia. Strains of S. aureus that exhibit the methicillin-resistant (MRSA) phenotype are predominant causes of healthcare-and community-acquired infections, including invasive disease in immune competent hosts, in immune suppression (e.g. neutropenia, solid-organ or bone marrow transplants), and in inherited immune dysfunctions manifesting recurring cutaneous infection (e.g. Job's Syndrome, Chronic Granulomatous Disease). The significant impact of MRSA on public health is of special concern in light of high rates of mortality associated with invasive S. aureus disease even with appropriate antimicrobial therapy (e.g. 15-40% in bacteremia and endocarditis). Increasing rates of life-threatening infections and decreasing susceptibility to antibiotics call for development of an effective vaccine targeting S. aureus.

Thus, there exists a need for effective immunogens that will provide host immune protection and passive immunoprotection against not only Candida and other immunogenically related pathogens but also Staphylococcus such as MRSA. The present invention satisfies these needs and provides related advantages as well.

### SUMMARY OF THE INVENTION

Peptides of the Candida albicans cell surface protein Als3 or Staphylococcus aureus collagen adhesin protein (CNA) as well as S. aureus clumping factor A as described herein are useful in immunizing against Candida or staphylococcal infections or both.

Accordingly, in a first aspect, the invention features an isolated polypeptide including the amino acid sequence of any one of SEQ ID NOs: 3-11, or a variant sequence thereof having up to three substitutions (e.g., conservative substitutions), deletions, or additions to the amino acid sequence of any one of SEQ ID NOs: 3-11, wherein the polypeptide does not include more than 20 contiguous amino acids of SEQ ID NO: 1 or SEQ ID NO:2 or SEQ ID NO:17. In a related aspect, the invention features an isolated polypeptide including the amino acid sequence of formula [ZNZPVSSBSFSYT]ₙ (Formula I), wherein B is an amino acid selected from the group consisting of D and E; Z is an amino acid selected from the group consisting of F, H, P, T, W, and Y; and n is an integer from 1 to 10; and wherein said polypeptide does not comprise more than 20 contiguous amino acids of SEQ ID NO: 1 or SEQ ID NO: 2 or SEQ ID NO:17. In a further related aspect, the invention features an isolated polypeptide including the amino acid sequence of formula [ZNUJVOOBUFOYT]ₙ (Formula II), wherein B is an amino acid selected from the group consisting of D and E; J is an amino acid selected from the group consisting of A, I, L, M, V, F, H, P, T, W, and Y; O is an amino acid selected from the group consisting of G, K, N, R, Q, S, T, Y, D, and E; U is an amino acid selected from the group consisting of D, E, S, T, and Y; Z is an amino acid selected from the group consisting of F, H, P, T, W, and Y; and n is an integer from 1 to 10; and wherein said polypeptide does not comprise more than 20 contiguous amino acids of SEQ ID NO: 1 or SEQ ID NO: 2 or SEQ ID NO:17. In some embodiments, the polypeptide includes the amino acid sequence of any one of SEQ ID NOs: 3-11. In some embodiments, the polypeptide consists of between 14 and 20 amino acids. In some embodiments, the N-terminal amino acid residue or C-terminal amino acid residue of the polypeptide is cysteine. In some embodiments, the amino acid sequence of the polypeptide includes or consists of the amino acid sequence of any one of SEQ ID NOs: 3-11. In certain embodiments, the isolated polypeptide is not or does not include any of the sequences of SEQ ID NOs: 13-16. In other embodiments, the polypeptide includes one or more sequences of SEQ ID NOs: 13-16 in addition to the sequence of SEQ ID NOs: 3-11, [ZNZPVSSBSFSYT]ₙ, [ZNUJVOOBUFOYT]ₙ, or variants thereof.

In a second aspect, the invention features an isolated conjugate including a polypeptide of the invention conjugated to a carrier. For example, the carrier may be keyhole limpet hemocyanin (KLH), CRM197, or tetanus toxoid or may be a phage, a yeast, a virus, virosome, or a recombinant virus-like particle. In some embodiments, the conjugate is a recombinant fusion protein.

In a third aspect, the invention features a vaccine including an immunogenic amount of a polypeptide or a conjugate of the invention, and a pharmaceutically acceptable excipient. In some embodiments, the vaccine includes a mixture of distinct polypeptides or conjugates of the invention. In some embodiments, the vaccine further includes an adjuvant, e.g., Alhydrogel. In some embodiments, a polypeptide or conjugate of the invention is produced synthetically or recombinantly. In some embodiments, the vaccine is for use in the vaccination of a mammal, e.g., a human, against candidiasis or a human against a staphylococcal infection or both. In some embodiments, the vaccine is to be administered by intramuscular, subcutaneous, or intradermal administration. In some embodiments, the vaccination further includes administering a booster dose. In some embodiments, the candidiasis is disseminated candidiasis, e.g., hematogenously disseminated candidiasis, or the candidiasis is mucosal candidiasis, or the candidiasis is caused by Candida albicans, Candida glabrata, Candida krusei, Candida parapsilosis, or Candida tropicalis.

In some embodiments, the staphylococcal infection is a systemic infection or is a SSTI infection such as an infection caused by MRSA.

In a fourth aspect, the invention features a method of vaccinating a mammal, e.g., a human, against candidiasis or against a staphylococcal infection including administering to the mammal the vaccine of the third aspect, thereby vaccinating the mammal against candidiasis or the staphylococcal infection. In some embodiments, the vaccine is administered by intramuscular, subcutaneous, or intradermal administration. In some embodiments, the administering further includes administering a booster dose. In some embodiments, the candidiasis is disseminated candidiasis, e.g., hematogenously disseminated candidiasis, or the candidiasis is mucosal candidiasis, or the candidiasis is caused by Candida albicans, Candida glabrata, Candida krusei, Candida parapsilosis, or Candida tropicalis. In some embodiments, the staphylococcal infection is a systemic infection or is a SSTI infection such as an infection caused by MRSA.

In a fifth aspect, the invention features a method of producing a chimeric vaccine including the steps of: (a) providing a phage, yeast, or virus; (b) inserting into the phage, yeast, or virus a nucleic acid molecule that encodes a polypeptide of the invention; (c) allowing expression of the polypeptide in the phage, yeast, or virus; (d) isolating the phage, yeast, or virus of step (c) including the expressed polypeptide; and (e) adding a pharmaceutically acceptable excipient to the isolated phage, yeast, or virus of step (d). In some embodiments, the polypeptide is displayed on the surface of the phage, yeast, or virus following step (c).

In a sixth aspect, the invention features an isolated monoclonal antibody that binds to a polypeptide or a conjugate of the invention. In some embodiments, the antibody is human or humanized, or is chimeric. In some embodiments, the antibody is produced recombinantly.

In a seventh aspect, the invention features a diagnostic composition including an antibody of the sixth aspect.

In an eighth aspect, the invention features a pharmaceutical composition including an antibody of the sixth aspect and a pharmaceutically acceptable excipient. In some embodiments, the pharmaceutical composition includes a mixture of antibodies of the sixth aspect with a plurality of distinct specificities.

In a ninth aspect, the invention features a pharmaceutical composition including polyclonal antibodies that bind to a polypeptide or a conjugate of the invention, or that bind to a mixture of distinct polypeptides or conjugates of the invention.

In some embodiments of the eighth or ninth aspect, the pharmaceutical composition is for use in the passive immunization of a mammal, e.g., a human, against candidiasis or against a staphylococcal infection or both. For example, the pharmaceutical composition may be administered by intramuscular, subcutaneous, or intradermal administration. In some embodiments, the candidiasis is disseminated candidiasis, e.g., hematogenously disseminated candidiasis, or the candidiasis is mucosal candidiasis, or the candidiasis is caused by Candida albicans, Candida glabrata, Candida krusei, Candida parapsilosis, or Candida tropicalis. In some embodiments, the staphylococcal infection is a systemic infection or is a SSTI infection such as an infection caused by MRSA.

In a tenth aspect, the invention features a method of passive immunization of a mammal, e.g., a human, against candidiasis or against a staphylococcal infection including administering to the mammal an effective amount of a pharmaceutical composition of the eighth or ninth aspect, thereby passively immunizing the mammal against the candidiasis or the staphylococcal infection or both. In some embodiments, the pharmaceutical composition is administered by intramuscular, subcutaneous, or intradermal administration. In some embodiments, the candidiasis is disseminated candidiasis, e.g., hematogenously disseminated candidiasis, or the candidiasis is mucosal candidiasis, or the candidiasis is caused by Candida albicans, Candida glabrata, Candida krusei, Candida parapsilosis, or Candida tropicalis. In some embodiments, the staphylococcal infection is a systemic infection or is a SSTI infection such as an infection caused by MRSA.

By “adjuvant” is meant one or more substances that cause stimulation of the immune system. In this context, an adjuvant is used to enhance an immune response to one or more vaccine antigens or antibodies. An adjuvant may be administered to a subject before, in combination with, or after administration of the vaccine or antibody. Examples of chemical compounds used as adjuvants include, but are not limited to, aluminum compounds (e.g., alum, Alhydrogel), oils, block polymers, immune stimulating complexes, vitamins and minerals (e.g., vitamin E, vitamin A, selenium, and vitamin B12), Quil A (saponins), bacterial and fungal cell wall components (e.g., lipopolysaccarides, lipoproteins, and glycoproteins), hormones, cytokines, and co-stimulatory factors.

By “antibody” is meant whole antibodies, immunoglobulins, or any antigen-binding fragment or single chains thereof. Antibodies, as used herein, can be mammalian (e.g., human or mouse), humanized, chimeric, recombinant, synthetically produced, or naturally isolated, and can be, e.g., monoclonal or polyclonal. In most mammals, including humans, whole antibodies have at least two heavy (H) chains and two light (L) chains connected by disulfide bonds. Each heavy chain consists of a heavy chain variable region (abbreviated herein as V_{H}) and a heavy chain constant region. The heavy chain constant region consists of three domains, C_{H}1, C_{H}2, and C_{H}3 and a hinge region between C_{H}1 and C_{H}2. Each light chain consists of a light chain variable region (abbreviated herein as V_{L}) and a light chain constant region. The light chain constant region consists of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four Elks, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies of the present invention include all known forms of antibodies and other protein scaffolds with antibody-like properties. For example, the antibody can be a human antibody, a humanized antibody, a bispecific antibody, a chimeric antibody, or a protein scaffold with antibody-like properties, such as fibronectin or ankyrin repeats. The antibody also can be a Fab, Fab′2, scFv, SMIP, diabody, nanobody, aptamers, or a domain antibody. The antibody can have any of the following isotypes: IgG (e.g., IgG1, IgG2, IgG3, and IgG4), IgM, IgA (e.g., IgA1, IgA2, and IgAsec), IgD, or IgE.

The term “antibody fragment,” as used herein, refers to one or more fragments of an antibody that retain the ability to specifically bind to an antigen. The antigen-binding function of an antibody can be performed by fragments of a full-length antibody, which include but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546 (1989)), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426 (1988) and Huston et al., Proc. Natl. Acad. Sci. USA 85:5879-5883 (1988)). These antibody fragments are obtained using conventional techniques known to those with skill in the art, and the fragments are screened for utility in the same manner as are intact antibodies. Antibody fragments can be produced by recombinant DNA techniques, or by enzymatic or chemical cleavage of intact immunoglobulins.

By “antigen” is meant a molecule to which an antibody can selectively bind. The target antigen may be a protein (e.g., an antigenic peptide), carbohydrate, nucleic acid, lipid, hapten, or other naturally occurring or synthetic compound. The target antigen may be a polypeptide or peptide mimic. An antigen may also be administered to an animal to generate an immune response in the animal.

By “carrier” in the context of a conjugate is meant a moiety or particle, e.g., KLH, CRM197, tetanus toxoid, a phage, a yeast, a virus, a virosome, or a recombinant virus-like particle, that is suitable for being linked to or displaying a polypeptide as described herein.

By “chimeric antibody” is meant an immunoglobulin or antibody whose variable regions derive from a first species and whose constant regions derive from a second species. Chimeric antibodies can be constructed, for example, by genetic engineering, from immunoglobulin gene segments belonging to different species (e.g., from a mouse and a human).

By “chimeric vaccine” is meant a vaccine that includes at least two distinct antigens, e.g., joined covalently. An example of a chimeric vaccine is a composition that includes a polypeptide displayed, e.g., on the surface of a particle such as a phage, virus, yeast, virosome, or recombinant virus-like particle.

By “conjugate” is meant a compound that includes a polypeptide of the invention linked to another moiety or particle, e.g., KLH, CRM197, tetanus toxoid, a phage, a yeast, a virus, a virosome, or a recombinant virus-like particle.

By “conservative substitution” in an amino acid sequence is meant replacement of an amino acid for another within a family of amino acids that are related in the chemical nature of their side chains.

Genetically encoded amino acids can be divided into four families: acidic (aspartate, glutamate); basic (lysine, arginine, histidine); nonpolar (alanine, valine, leucine, isoleucine, proline, phenylalanine, methionine, tryptophan); and uncharged polar (glycine, asparagine, glutamine, cysteine, serine, threonine, tyrosine). Phenylalanine, tryptophan, and tyrosine are sometimes grouped as aromatic amino acids. In similar fashion, the amino acids can also be separated into the following groups: acidic (aspartate, glutamate); basic (lysine, arginine, histidine); alipathic (glycine, alanine, valine, leucine, isoleucine, serine, threonine), with serine and threonine optionally grouped separately as alipathic-hydroxyl; aromatic (phenylalanine, tyrosine, tryptophan); amide (asparagine, glutamine); and sulfur-containing (cysteine, methionine).

Whether a change in the amino acid sequence results in a functional variant can be determined by assessing the ability of the variant polypeptide to function in a fashion similar to the wild-type polypeptide using standard methods such as those described herein.

By “diagnostic composition” is meant a composition containing a polypeptide, conjugate, vaccine, or antibody of the invention, formulated for use in conjunction with a diagnostic method.

By “effective amount” in the context of passive immunization using a pharmaceutical composition, e.g., comprising an antibody, is meant the amount of the pharmaceutical composition required to passively immunize in a clinically relevant manner An effective amount of pharmaceutical composition used to practice the methods of passive immunization described herein varies depending upon the manner of administration, the age, body weight, and general health of the subject. Ultimately, the prescribers will decide the appropriate amount and dosage regimen.

By “flanking amino acid” is meant an amino acid in a polypeptide sequence that is immediately adjacent to the N- or C-terminus of a particular defined sequence. Desirably, a flanking amino acid is present on the N- and/or C-terminus of the amino acid sequence of SEQ ID NO: 1 or 2 or a fragment thereof; and more desirably, 1, 2, 3, 4, 5, 6, 7, 8, 9, or 10 flanking amino acids are present at the N- and/or C-terminus of the amino acid sequence of SEQ ID NO: 1 or 2 or 17, or fragment thereof.

By “fusion protein” is meant a protein that includes a polypeptide of the invention, e.g., a peptide fragment or variant, and a fusion partner.

By “fusion partner” is meant a heterologous sequence that can be fused to a polypeptide or peptide of the invention, e.g., one or more of Peptide 3-11 or variants thereof. Examples of fusion partners are described herein and include detection markers, stabilizing domains, sequences which aid in production or purification of the protein, or domains which increase the antigenicity of the polypeptide.

By “Als3 polypeptide” is meant, in general, the polypeptide having the amino acid sequence of SEQ ID NO: 1. In some instances, an Als3 protein has substantial identity to SEQ ID NO:1.

By “collagen adhesin protein of Staphylococcus aureus” is meant the polypeptide having the amino acid sequence of SEQ ID NO:2. In some instances, a collagen adhesin protein of Staphylococcus aureus protein has substantial identity to SEQ ID NO:2.

By “clumping factor A protein (clfA) of Staphylococcus aureus” is meant the polypeptide having the amino acid sequence of SEQ ID NO:17. In some instances, a clumping factor A protein of Staphylococcus aureus protein has substantial identity to SEQ ID NO:17.

By “immunogenic” is meant any substance that is capable of inducing an immune response in a subject.

By “immunogenic amount” in the context of a vaccine is meant an amount of the vaccine required to induce an immune response in a subject in a clinically relevant manner. An immunogenic amount of vaccine used to practice the methods of vaccination as described herein varies depending upon the manner of administration, the age, body weight, and general health of the subject. Ultimately, the prescribers will decide the appropriate amount and dosage regimen.

By “isolated” or “purified” is meant separated from other naturally accompanying components. Typically, a compound (e.g., nucleic acid, polypeptide, antibody, or small molecule) is substantially isolated when it is at least 60%, by weight, free from the proteins and/or naturally occurring organic molecules with which it is naturally associated. The definition also extends, e.g., to a polypeptide or nucleic acid molecule separated from its flanking sequences (e.g., for an amino acid sequence, isolated refers to a sequence that is free from the flanking amino acids with which the sequence is naturally associated in a polypeptide). In some instances, the compound is at least 75%, more preferably at least 90%, and most preferably at least 99%, by weight, isolated. An isolated compound, e.g., polypeptide, may be obtained by standard techniques, for example, by extraction from a natural source (e.g., purification from a cell infected with Candida); by expression of a recombinant nucleic acid encoding an Als3 or CNA fragment or variant, or a fusion protein thereof; or by chemically synthesizing the polypeptide. Purity can be measured by any appropriate method, e.g., by column chromatography, polyacrylamide gel electrophoresis, or HPLC analysis.

By “linked to” or “conjugated to” in the context of a conjugate is meant a covalent or non-covalent interaction between the polypeptide and the carrier or fusion partner. Non-covalent interactions include, but are not limited to, hydrogen bonding, ionic interactions among charged groups, electrostatic binding, van der Waals interactions, hydrophobic interactions among non-polar groups, lipophobic interactions, and LogP-based attractions.

By “monoclonal antibody” is meant an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique and those described herein such as, for example, a hybridoma method, as described by Kohler et al., Nature 256:495 (1975), a transgenic animal (e.g., Lonberg et al., Nature 368(6474):856-859 (1994)), recombinant DNA methods (e.g., U.S. Pat. No. 4,816,567), or using phage, yeast, or synthetic scaffold antibody libraries using the techniques described in, for example, Clackson et al., Nature 352:624-628 (1991) and Marks et al., J. Mol. Biol. 222:581-597 (1991).

By “nucleic acid molecule” is meant a molecule, e.g., RNA or DNA, having a sequence of two or more covalently bonded, naturally occurring or modified nucleotides. The nucleic acid molecule may be, e.g., single or double stranded, and may include modified or unmodified nucleotides, or mixtures or combinations thereof. Various salts, mixed salts, and free acid forms are also included.

By “patient” or “subject” is meant a mammal, including, but not limited to, a human or non-human mammal, such as a bovine, equine, canine, ovine, or feline.

The terms “peptide,” “polypeptide,” and “protein” are used interchangeably and refer to any chain of two or more natural or unnatural amino acids, regardless of post-translational modification (e.g., glycosylation or phosphorylation), constituting all or part of a naturally-occurring or non-naturally occurring polypeptide or peptide, as is described herein.

As used herein, a natural amino acid is a natural a-amino acid having the L-configuration, such as those normally occurring in natural polypeptides. Unnatural amino acid refers to an amino acid that normally does not occur in polypeptides, e.g., an epimer of a natural α-amino acid having the L configuration, that is to say an amino acid having the unnatural D-configuration; or a (D,L)-isomeric mixture thereof; or a homolog of such an amino acid, for example, a β-amino acid, an α,α-disubstituted amino acid, or an α-amino acid wherein the amino acid side chain has been shortened by one or two methylene groups or lengthened to up to 10 carbon atoms, such as an a-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

The terms “pharmaceutically acceptable carrier” and “pharmaceutically acceptable excipient” are used interchangeably and mean a carrier or excipient that is physiologically acceptable to the treated patient while retaining the therapeutic properties of the compound with which it is administered. One exemplary pharmaceutically acceptable carrier substance is physiological saline. Other physiologically acceptable carriers and their formulations are known to those skilled in the art and described, for example, in Remington's Pharmaceutical Sciences, (20^{th} edition), ed. A. Gennaro, 2000, Lippincott, Williams & Wilkins, Philadelphia, Pa.

By “pharmaceutical composition” is meant a composition containing a polypeptide, conjugate, vaccine, or antibody of the invention, formulated with a pharmaceutically acceptable excipient, and manufactured or sold with the approval of a governmental regulatory agency as part of a therapeutic regimen for the treatment or prevention of a disease or event in a mammal. Pharmaceutical compositions can be formulated, for example, for intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

By “specifically binds” is meant the preferential association of a binding moiety (e.g., an antibody, antibody fragment, receptor, ligand, or small molecule portion of an agent as described herein) to a target molecule (e.g., a polypeptide or conjugate including same) or to a cell or tissue bearing the target molecule (e.g., a cell surface antigen, such as a receptor or ligand) and not to non-target molecules, cells, or tissues lacking the target molecule. It is recognized that a certain degree of non-specific interaction may occur between a binding moiety and a non-target molecule (present alone or in combination with a cell or tissue). Nevertheless, specific binding may be distinguished as mediated through specific recognition of the target molecule. Specific binding results in a stronger association between the binding moiety (e.g., an antibody) and the target molecule (e.g., a polypeptide or conjugate including same) than between the binding moiety and, e.g., non-target molecules or other compositions lacking the target molecule. Specific binding typically results in greater than 2-fold, preferably greater than 5-fold, more preferably greater than 10-fold and most preferably greater than 100-fold increase in amount of bound binding moiety (per unit time) to e.g., a cell or tissue bearing the target molecule or marker as compared to a cell or tissue lacking that target molecule or marker. Binding moieties bind to the target molecule or marker with a dissociation constant of e.g., less than 10⁻⁶M, less than 10⁻⁷M, 10⁻⁸M, 10⁻⁹M, 10⁻¹⁰M, 10⁻¹¹M, or 10⁻¹²M, or even less than 10⁻¹³M, 10⁻¹⁴M, or 10⁻¹⁵M. Specific binding to a protein under such conditions requires a binding moiety that is selected for its specificity for that particular protein. A variety of assay formats are appropriate for selecting binding moieties (e.g., antibodies) capable of specifically binding to a particular target molecule. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

By “substantially identical” is meant an amino acid sequence or nucleic acid sequence that exhibits at least 50% identity to a reference sequence. Such a sequence is generally at least, e.g., 50%, 60%, 70%, 75%, 80%, 85%, 90%, 95%, 96%, 97%, 98%, or 99% identical at the amino acid level or nucleic acid level to a reference sequence. In general, for polypeptides, the length of comparison sequences can be at least five amino acids, e.g., 10, 20, 30, 40, 50, 60, 70, 80, 90, 100, 125, 150, 175, 200, 250, 300, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, or more nucleotides, up to the entire length of the nucleic acid molecule. It is understood that for the purposes of determining sequence identity when comparing a DNA sequence to an RNA sequence, a thymine nucleotide is equivalent to a uracil nucleotide.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al., J. Mol. Biol. 215: 403-10, 1990). These and other alignment algorithms are accessible using publicly available computer software such as “Best Fit” (Smith and Waterman, Advances in Applied Mathematics, 482-489, 1981) as incorporated into GeneMatcher Plus™ (Schwarz and Dayhof, Atlas of Protein Sequence and Structure, Dayhoff, M. O., Ed pp 353-358, 1979), BLAST, BLAST-2, BLAST-P, BLAST-N, BLAST-X, WU-BLAST-2, ALIGN, ALIGN-2, CLUSTAL, or Megalign (DNASTAR). In addition, those skilled in the art can determine appropriate parameters for measuring alignment, including any algorithms needed to achieve optimal alignment over the length of the sequences being compared.

By “Staphylococcus aureus skin or soft tissue infection”, “Staphylococcus aureus SSTI”, “Staphylococcus aureus skin/skin structure infection”, and “Staphylococcus aureus SSSI” are used interchangeably herein and refer to a skin or soft tissue infection (e.g. cellulitis, soft tissue abscess, dermonecrosis, myositis, or other infections) resulting from S. aureus entering the body at a site where a cut, scrape, bite, or other wound has broken the skin. In some instances, S. aureus SSSI is the result of S. aureus living on the body, and may occur spontaneously in the absence of a visible site of skin injury or wound. Such infections may affect the layers of the skin or deeper tissues, such as muscle and connective tissue (the interlacing framework of tissue that forms ligaments, tendons, and other supporting structures of the body). Skin abscesses may also occur in areas of the skin where the body has been fighting a S. aureus infection. The more important strains of S. aureus responsible for skin or soft tissue infections are the antibiotic-resistant Staphylococcus known as methicillin-resistant Staphylococcus aureus (MRSA); vancomycin-resistant and daptomycin-resistant strains of S. aureus may also cause SSSI. MRSA is resistant to commonplace antibiotics. Staphylococcus aureus SSSIs may also be caused by methicillin-sensitive Staphylococcus aureus (MSSA).

Mammals which are at risk of developing a S. aureus skin or soft tissue infection can be treated in a prophylactic mode. Alternatively, mammals may be treated when presenting with symptoms of a S. aureus skin or soft tissue infection. Vaccination as described herein will reduce the severity, delay, or prevent the development of symptoms. Mammals are at elevated risk of infection if they are hospitalized or living in an institutionalized community, antibiotic treated, or immunosuppressed including children having HIV/AIDS or other diseases that compromise immune function, individuals having frequent contact with the healthcare system, having a chronic illness such as diabetes, cancer, HIV/AIDS, being very young or very old, frequent use of antibiotics, having an open wound, dermatitis or skin lesions, poor nutrition or poor hygiene. Other mammals at risk include those living in crowded living conditions, military personnel, especially deployed troops, athletes, and prison inmates. Still others at risk of developing a S. aureus skin or soft tissue infection are those individuals previously having such infections or individuals scheduled for or having had a surgical or invasive medical procedure.

A “target molecule” or “target cell” is meant a molecule (e.g., a polypeptide, epitope, antigen, receptor, or ligand) or cell to which a binding moiety (e.g., an antibody) can specifically bind. In some instances, target molecules are exposed on the exterior of a target cell (e.g., a cell surface or secreted protein), but target molecules may alternately or also be present in the interior of a target cell.

By “treating” or “treatment” is meant the medical management of a patient with the intent to cure, ameliorate, stabilize, reduce the likelihood of, or prevent a disease, pathological condition, disorder, or event, by administering a pharmaceutical composition. This term includes active treatment, that is, treatment directed specifically toward the improvement or associated with the cure of a disease, pathological condition, disorder, or event, and also includes causal treatment, that is, treatment directed toward removal of the cause of the associated disease, pathological condition, disorder, or event. In addition, this term includes palliative treatment, that is, treatment designed for the relief of symptoms rather than the curing of the disease, pathological condition, disorder, or event; symptomatic treatment, that is, treatment directed toward constitutional symptoms of the associated disease, pathological condition, disorder, or event; preventative treatment, that is, treatment directed to minimizing or partially or completely inhibiting the development of the associated disease, pathological condition, disorder, or event, e.g., in a patient who is not yet ill, but who is susceptible to, or otherwise at risk of, a particular disease, pathological condition, disorder, or event; and supportive treatment, that is, treatment employed to supplement another specific therapy directed toward the improvement of the associated disease, pathological condition, disorder, or event.

By “vaccine,” as used herein, is meant a composition that elicits an immune response in a subject to which it is administered.

By “vaccinate,” as used herein, is meant to treat a patient by administering a vaccine, e.g., to prevent or ameliorate a disease, pathological condition, disorder, or event.

By “variant” in the context of a polypeptide or portion thereof as described herein, or a nucleic acid molecule encoding same, is meant to include substitutions or alterations in the amino acid sequence or nucleic acid sequence, e.g., resulting in a substantially identical sequence. A polypeptide having a variant sequence may maintain at least one biological activity of the original polypeptide, e.g., immunogenic activity. The term “variant” includes, e.g., amino acid insertional derivatives such as amino and/or carboxylterminal fusions, as well as intrasequence insertions of single or multiple amino acids. Insertional amino acid variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue inserted in its place. Where the protein is derivatized by amino acid substitution, amino acids are generally replaced by conservative substitutions, e.g., other amino acids having similar physical chemical properties such as hydrophobicity, hydrophilicity, electronegativity, bulky sidechains and the like.

For purposes of the present invention, variants also include single or multiple substitutions, deletions and/or additions of any component(s) naturally or artificially associated with the portion of a naturally occurring protein from which the polypeptide may be derived, such as carbohydrate, lipid and/or other proteinaceous moieties. All such molecules are encompassed by the term “variant.”

By “variant sequence” is meant the amino acid or nucleic acid sequence of a variant as defined herein.

Other features and advantages of the invention will be apparent from the following Detailed Description and the claims

### DETAILED DESCRIPTION OF THE INVENTION

Candida albicans and Staphylococcus aureus are common pathogens in humans. The identification of the Als3 and CNA and clfA fragments and other compositions described herein allow, e.g., for the effective treatment of and vaccination against candidiasis or staphylococcus infections or both.

The invention provides polypeptides, e.g., derived from Als3 or CAN or clumping factor A, conjugates, vaccines, antibodies, compositions, methods of vaccination using same, and methods of production of same, as described in further detail below.

### Polypeptides

The invention features polypeptides derived from Als3 (SEQ ID NO: 1) or CNA(SEQ ID NO: 2) or clumping factor A (SEQ ID NO17), e.g., including the amino acid sequence of any one of SEQ ID NOs: 3-11, or a variant sequence thereof having zero, one, two, or three substitutions, deletions, or additions to the amino acid sequence of any one of SEQ ID NOs: 3-11, wherein the polypeptide does not include more than 20 contiguous amino acids of SEQ ID NO: 1 or SEQ ID NO: 2 or SEQ ID NO:17.

| SEQ ID NO: 1 is an amino acid sequence of C. albicans Als3: | |
|---|---|
| 1 | mllqflllsl cvsvatakvi tgvfnsfdsl twtragnyay kgpnrptwna vlgwsldgts |
| 61 | anpgdtftln mpcvfkfitd qtsvdltadg vkyatcqfys geefttfssl kctvsntlts |
| 121 | sikalgtvtl pisfnvggtg slvdlesskc fkagtntvtf ndgdkkisid vdfektneda |
| 181 | sgyfiasrli psinkasity vapqcangyt sgamgftigs gdttidcsnv hvgitkglnd |
| 241 | wnfpvssdsl synktcsstg isityenvpa gyrpffdvyt svsdqnrqlk ytndyacvgs |
| 301 | slqskpfnlr lrgynnsean sngfvivatt rtvtdsttav ttlpfnpsid ktktieilqp |
| 361 | iptttittsy vgvttsystk tapigetatv ivdvpyhttt tvtsewtgti tttttrtnpt |
| 421 | dsidtvvvqv pspnptvstt eywsqsyatt ttitappggt dtviireppn htvttteyws |
| 481 | qsyattttvt appggtdsvi ireppnptvt tteywsqsya ttttitappg gtdtviirep |
| 541 | pnhtvtttey wsqsyatttt vtappggtds viireppnpt vttteywsqs ytttttvtap |
| 601 | pggtdsviir eppnptvttt eywsqsyatt ttvtatpggt dtviireppn ytvttteyws |
| 661 | qsyattttvt appggtdtvi ireppnytvt tteywsqsya ttttvtappg gtdtviirep |
| 721 | pnytvtttey wsqsyattst itappggtdt viireppnyt vttteywsqs yattttvtgp |
| 781 | pggtdtviir eppsptvttt eywsqsyatt ttvtappggt atviikeppn ytvttteyws |
| 841 | qsyattttvt gppggtdtvi ireppnptvt tteywsqsfa ttttvtappg gtdsviirep |
| 901 | pnptvtttey wsqsyatttt vtappggtds viireppnpt vttteywsqs ytttttvtap |
| 961 | pggtdsviir eppnptvttt eywsqsyatt ttvtatpggt dtviireppn ytvttteyws |
| 1021 | qsyattttvt appggtdtvi ireppnytvt tteywsqsya ttttvtappg gtdtviirep |
| 1081 | pnhtvtttey wsqsyttttt viappggtds viireppnpt vttteywsqs yattttitap |
| 1141 | pgetdtvlir eppnhtvttt eywsqsfatt ttvtappggt dtviireppn htvttteyws |
| 1201 | qsyattttit appgetdtvl ireppnhtvt tteywsqsya ttttiiappg etdtvlirep |
| 1261 | pnptvtttey wsqsyttatt vtappggtdt viiydtmsss eissfsrphy tnhttlwstt |
| 1321 | wvietktite tscegdkgcs wvsystrivt ipnnietpmv tntvdtttte stlqspsgif |
| 1381 | sesgvsvete sstfttaqtn psvpttesev vfttkgnngn gpyespstnv kssmdensef |
| 1441 | ttstaastst dienetiatt gsveasspii sssadetttv tttaestsvi eqqtnnnggg |
| 1501 | napsatstst sspsttttan ndsvitstts inqsqsqsns dtqqttlsqq mtsslvslhm |
| 1561 | lttfdgsgsv ighstwlcgl itllslfi |
| SEQ ID NO: 2 is an amino acid sequence of S. aureus CNA: | |
| 1 | mnknvlkfmv fimllniitp lfnkneafaa rdisstnvtd ltvspskied ggkttvkmtf |
| 61 | ddkngkiqng dmikvawpts gtvkiegysk tvpltvkgeq vgqavitpdg atitfndkve |
| 121 | klsdvsgfae fevqgrnltq tntsddkvat itsgnkstnv tvhkseagts svfyyktgdm |
| 181 | lpedtthvrw flninneksy vskditikdq igggqqldls tlninvtgth snyysgqsai |
| 241 | tdfekafpgs kitvdntknt idvtipqgyg synsfsinyk tkitneggke fvnnsgawyq |
| 301 | ehgkeevngk sfnhtvhnin anagiegtvk gelkvlkqdk dtkapianvk fklskkdgsv |
| 361 | vkdngkeiei itdangiani kalpsgdyil keieaprpyt fdkdkeypft mkdtdnqgyf |
| 421 | ttienakaie ktkdvsaqkv wegtqkvkpt iyfklykqdd nqnttpvdka eikkledgtt |
| 481 | kvtwsnlpen dkngkaikyl vkevnagged ttpegytkke nglvvtntek piettsisge |
| 541 | kvwddkdnqd gkrpekvsvn llangekvkt ldvtsetnwk yefkdlpkyd egkkieytvt |
| 601 | edhvkdyttd ingttitnky tpgetsatvt knwddnnnqd gkrpteikve lygdgkatgk |
| 661 | tailnesnnw thtwtgldek akgqqvkytv eeltkvkgyt thvdnndmgn livtnkytpe |
| 721 | ttsisgekvw ddkdnqdgkr pekvsvnlla dgekvktldv tsetnwkyef kdlpkydegk |
| 781 | kieytvtedh vkdyttding ttitnkytpg etsatvtknw ddnnnqdgkr pteikvelyq |
| 841 | dgkatgktai lnesnnwtht wtgldekakg qqvkytveel tkvkgytthv dnndmgnliv |
| 901 | tnkytpetts isgekvwddk dnqdgkrpek vsvnllange kvktldvtse tnwkyefkdl |
| 961 | pkydegkkie ytvtedhvkd yttdingtti tnkytpgets atvtknwddn nnqdgkrpte |
| 1021 | ikvelygdgk atgktailne snnwthtwtg ldekakgqqv kytvdeltkv ngytthvdnn |
| 1081 | dmgnlivtnk ytpkkpnkpi ypekpkdktp ptkpdhsnkv kptppdkpsk vdkddqpkdn |
| 1141 | ktkpenplke lpktgmkiit switwvfigi lglylilrkr fns |
| SEQ ID NO: 17 is an amino acid sequence of S. aureus clfA: | |
| 1 | mnmkkkekha irkksigvas vlvgtligfg llsskeadas ensvtqsdsa snesksndss |
| 61 | sysaapktdd tnvsdtktss ntnngetsva qnpaggettg ssstnattee tpvtgeattt |
| 121 | ttnqantpat tqssntnaee lvngtsnett sndtntvssv nspqnstnae nvsttqdtst |
| 181 | eatpsnnesa pqntdasnkd vvsqavnpst prmrafslaa vaadapaagt ditngltdvk |
| 241 | vtidsgttvy phgagyvkln ygfsvpnsav kgdtfkitvp kelnlngvts takvppimag |
| 301 | dqvlangvid sdgnviytft dyvdnkenvt anitmpayid penvtktgnv tlttgigtnt |
| 361 | asktvlidye kyggfhnlsi kgtidgidkt nntyrqtiyv npsgdnvvlp altgnlipnt |
| 421 | ksnalidakn tdikvyrvdn andlsesyyv npsdfedvtn qvrisfpnan qykvefptdd |
| 481 | dgittpyivv vnghidpast gdlalrstfy gydsnfiwrs mswdnevafn ngsgsgdgid |
| 541 | kpvvpeqpde pgeiepiped sdsdpgsdsg sdsnsdsgsd sgsdstsdsg sdsasdsdsa |
| 601 | sdsdsasdsd sasdsdsasd sdsasdsdsa sdsdsasdsd sasdsdsasd sdsasdsdsa |
| 661 | sdsdsasdsd sdsdsdsdsd sdsdsdsdsd sdsdsdsdsd sdsdsdsdsd sdsdsdsdsd |
| 721 | sdsdsdsdsd sdsdsdsdsd sdsdsdsdsd sdsdsdsdsd sdsdsdsdsd sdsdsdsdsd |
| 781 | sdsdsdsdsd sdsdsdsdsd sdsdsdsdsd sdsdsdsdsd sasdsdsdsd sesdsdsdsd |
| 841 | sdsdsdsdsd sdsdsesdsd sdsdsdsesd sdsdsdsdsd sasdsdsgsd sdsssdsdsd |
| 901 | stsdtgsdnd sdsdsnsdse sgsnnnvvpp nspkngtnas nkneakdske plpdtgsede |
| 961 | antsliwgll aslgslllfr rkkenkdkk |

SEQ ID NOs: 3-11 are 9-mer fragments of the amino acid sequence of Als1
or CAN or clfA, as shown in Table 1.

TABLE 1

| | SEQ ID | |
|---|---|---|
| Peptide | NO | Sequence |
| 1 | 3 | PVSSDSLSY |
| 2 | 4 | SSDSLSYNK |
| 3 | 5 | VTDSTTAVT |
| 4 | 6 | VTSEWTGTI |
| 5 | 7 | PTDSIDTVV |
| 6 | 8 | STNVTDLTV |
| 7 | 9 | VTSETNWKY |
| 8 | 10 | VTEDHVKDY |
| 9 | 11 | PTEIKVELY |

Additional polypeptides may include a sequence of the general formula (I) [ZNZPVSSBSFSYT]ₙ (SEQ ID NO: 12), wherein B is an amino acid selected from the group consisting of D and E; Z is an amino acid selected from the group consisting of F, H, P, T, W, and Y; and n is an integer from 1 to 10 or of general formula (II) [ZNUJVOOBUFOYT]ₙ, wherein B is an amino acid selected from the group consisting of D and E; J is an amino acid selected from the group consisting of A, I, L, M, V, F, H, P, T, W, and Y; O is an amino acid selected from the group consisting of G, K, N, R, Q, S, T, Y, D, and E; U is an amino acid selected from the group consisting of D, E, S, T, and Y; Z is an amino acid selected from the group consisting of F, H, P, T, W, and Y; and n is an integer from 1 to 10.

The polypeptides of Table 1 or other polypeptides described herein may have a variant or otherwise modified amino acid sequence. For example, in variants of the polypeptides of Table 1, each substitution, deletion, or addition, if any, may be made, e.g., at position 1, 2, 3, 4, 5, 6, 7, 8, 9 or at the N- or C-terminal end of the polypeptide.

In some instances, the polypeptide is between 14 and 20 amino acids, e.g., 10, 11, 12, 13, 14, 15, 16, 17, 18, 19, or 20 amino acids. In other instances, the polypeptide is shorter than 14 amino acids, e.g., 10, 11, 12, or 13 amino acids or even 8 or 7 amino acids. The polypeptide may be longer than 20 amino acids provided that it does not include more than 20 contiguous amino acids of SEQ ID NO: 1 or SEQ ID NO: 2 or SEQ ID NO:17.

In some instances, the polypeptide is not or does not include a sequence of SEQ ID NOs: 13-16, as shown in Table 2. Alternatively, the polypeptide includes one or more sequences of SEQ ID NOs: 13-16 in addition to the sequence of SEQ ID NOs: 3-12, [ZNUJVOOBUFOYT]ₙ, or variants thereof.

TABLE 2

| SEQ ID | |
|---|---|
| NO | Sequence |
| 13 | KGLNDWNYPVSSESFSYT |
| 14 | KGLNDWNYPVSSESFSY |
| 15 | KGLNDWNYPVSSESFS |
| 16 | WNYPVSSES |

In some instances, a modification to a polypeptide as described herein does not substantially reduce the biological activity, e.g., immunogenic activity, of the polypeptide. The modified polypeptide may have or may optimize a characteristic of a polypeptide, such as in vivo stability, bioavailability, toxicity, immunological activity, immunological identity, or conjugation properties.

Modifications include those by natural processes, such as posttranslational processing, or by chemical modification techniques known in the art. Modifications may occur anywhere in a polypeptide including the polypeptide backbone, the amino acid side chains, and the amino- or carboxy-terminus The same type of modification may be present in the same or varying degrees at several sites in a given polypeptide, and a polypeptide may contain more than one type of modification.

A variant or otherwise modified polypeptide can also include one or more amino acid insertions, deletions, or substitutions, either conservative or non-conservative (e.g., D-amino acids, desamino acids) in the polypeptide sequence. For example, the addition of one or more cysteine residues to the amino or carboxy terminus of any of the polypeptides of the invention can facilitate conjugation of these polypeptides. Exemplary polypeptides having an N- or C-terminal cysteine.

Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically, e.g., using methods known in the art, can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Variants may be generated by substitutional mutagenesis and retain or even increase the biological activity, e.g., immunogenic activity, of the original polypeptide.

The polypeptides described herein can be obtained, e.g., by chemical synthesis using a commercially available automated peptide synthesizer. The synthesized protein or polypeptide can be precipitated and further purified, for example by high performance liquid chromatography (HPLC). Alternatively, the proteins and polypeptides can be obtained by recombinant methods, e.g., that are well-known in the art.

### Conjugates

Polypeptides of the invention may be conjugated to another moiety or particle.

Protein Moieties

In some instances, it may be useful to conjugate the polypeptide to a protein that is immunogenic in the species to be immunized, e.g., keyhole limpet hemocyanin (KLH), CRM197, tetanus toxoid, diptheria toxoid, serum albumin, bovine thyroglobulin, soybean trypsin inhibitor, or a polycation (poly-L-Lysine or poly-L-arginine), e.g., using a bifunctional or derivatizing agent as known in the art, for example, maleimidobenzoyl sulfosuccinimide ester (conjugation through cysteine residues), N-hydroxysuccinimide (through lysine residues), glutaraldehyde, or succinic anhydride.

In some instances, the conjugate may be a recombinant fusion protein, e.g., to facilitate expression and purification of the polypeptide.

Particles for Conjugation or Display of Polypeptides

In some instances, polypeptides are conjugated to or displayed on a particle, e.g., a phage, a yeast, a virus, a virosome, or a recombinant virus-like particle.

For example, one or more polypeptides may be conjugated to a phage, a yeast, or a virus particle, e.g., to the surface of the particle. In one embodiment, a nucleic acid molecule encoding the polypeptide is inserted into the phage, yeast, or virus particle, resulting in expression of the polypeptide in the phage, yeast, or virus, e.g., at the surface of the particle. The phage, yeast, or virus population containing the polypeptide may then be isolated and prepared, e.g., as a vaccine, by adding a pharmaceutically acceptable excipient.

In some embodiments, polypeptides as described herein are conjugated to a virosome or virus-like particle (VLP). Virosomes and VLPs generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. Viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Q.beta.-phage (such as coat proteins), GA-phage, fr-phage, AP205 phage, and Ty (such as retrotransposon Ty protein p 1). Virosomes are discussed further in, e.g., Gluck et al. (2002), Vaccine 20:B10-B16, which is incorporated by reference in its entirety.

VLPs are discussed further, e.g., in Niikura et al. (2002), Virology 293:273-280; Lenz et al. (2001), J Immunol 166:5346-5355; Pinto et al. (2003), J Infect Dis 188:327-338; Gerber et al. (2001), Viral 75:4752-4760; WO03/024480; and WO03/024481, each of which is incorporated by reference in its entirety.

### Antibodies

The invention features monoclonal and polyclonal antibodies that bind to the polypeptides or conjugates described herein.

Monoclonal Antibodies

Monoclonal antibodies may be made, e.g., using the hybridoma method first described by Kohler et al., Nature 256:495, 1975, or may be made by recombinant DNA methods (see, e.g., U.S. Pat. No. 4,816,567). In the hybridoma method, a mouse or other appropriate host animal, such as a hamster or macaque monkey, is immunized, e.g., using a polypeptide or conjugate described herein, to elicit lymphocytes that produce or are capable of producing antibodies that will specifically bind to the polypeptide or conjugate used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103, Academic Press, 1986).

The hybridoma cells thus prepared are seeded and grown in a suitable culture medium that can contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Exemplary myeloma cells are those that fuse efficiently, support stable high-level production of antibody by the selected antibody-producing cells, and are sensitive to a medium such as HAT medium. Among these, particular myeloma cell lines that may be considered for use are murine myeloma lines, such as those derived from MOPC-21 and MPC-11 mouse tumors available from the Salk Institute Cell Distribution Center, San Diego, Calif., USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol. 133:3001, 1984; Brodeur et al., Monoclonal Antibody Production Techniques and Applications, pp. 51-63, Marcel Dekker, Inc., New York, 1987).

Culture medium in which hybridoma cells are growing is assayed for production of monoclonal antibodies directed against the antigen. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, clones may be subcloned by limiting dilution procedures and grown by standard methods (Goding, Monoclonal Antibodies: Principles and Practice, pp. 59-103, Academic Press, 1986). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described, for example, in McCafferty et al., Nature 348:552-554, 1990.

Clackson et al., Nature 352:624-628, 1991 and Marks et al., J. Mol. Biol. 222:581-597, 1991, describe the isolation of murine and human antibodies, respectively, using phage libraries. Subsequent publications describe the production of high affinity (nM range) human antibodies by chain shuffling (Marks et al., Bio/Technology 10:779-783, 1992), as well as combinatorial infection and in vivo recombination as a strategy for constructing very large phage libraries (Waterhouse et al., Nucl. Acids. Res. 21:2265-2266, 1993). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison et al., Proc. Natl. Acad. Sci. U.S.A. 81:6851, 1984), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Polyclonal Antibodies

Polyclonal antibodies are typically raised in animals by multiple injections, e.g., subcutaneous or intraperitoneal injections, of the relevant antigen and an adjuvant. In some instances, it may be useful to conjugate the polypeptide to a protein that is immunogenic in the species to be immunized, e.g., keyhole limpet hemocyanin (KLH), CRM197, tetanus toxoid, diptheria toxoid, serum albumin, bovine thyroglobulin, soybean trypsin inhibitor, or a polycation (poly-L-Lysine or poly-L-arginine), e.g., using a bifunctional or derivatizing agent as known in the art, for example, maleimidobenzoyl sulfosuccinimide ester (conjugation through cysteine residues), N-hydroxysuccinimide (through lysine residues), glutaraldehyde, or succinic anhydride.

### Vaccines and Antibody-Containing Pharmaceutical Compositions

Formulations for vaccines and antibody-containing pharmaceutical compositions (collectively “compositions”) as described herein can be prepared using standard pharmaceutical formulation chemistries and methodologies that are readily available to the reasonably skilled artisan. For example, polypeptides, conjugates, or antibodies as described herein can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions may include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e.g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions that are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Alternatively, the polypeptides, conjugates, and antibodies described herein may be encapsulated, adsorbed to, or associated with particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated compositions will include an amount of one or more polypeptides or conjugates described herein that is sufficient to mount an immunological response. An immunogenic amount can be readily determined by one of skill in the art. Such an amount will fall in a relatively broad range that can be determined through routine trials. The compositions may contain from about 0.1% to about 99.9% of the polypeptides, conjugates, or antibodies, and can be administered directly to the subject or, alternatively, delivered ex vivo, to cells derived from the subject, using methods known to those skilled in the art.

Compositions can include a mixture of distinct polypeptides, conjugates, or antibodies as described herein. For example, vaccines may include, e.g., 2, 3, 4, 5, 6, 7, 8, or more distinct polypeptides or conjugates as described herein, e.g., containing or consisting of the amino acid sequences of SEQ ID NOs: 3-11, or a variant sequence thereof having up to three substitutions (e.g., conservative substitutions), deletions, or additions to the amino acid sequence of any one of SEQ ID NOs: 3-11 or a sequence of formula (I) or formula (II). In one embodiment, a vaccine includes nine distinct polypeptides, wherein the amino acid sequence of the nine polypeptides consist of the sequence of SEQ ID NOs: 3-11. In another embodiment, antibody-containing pharmaceutical compositions may include a mixture of monoclonal or polyclonal antibodies, e.g., having distinct specificities to polypeptides or conjugates as described herein.

Substances that stimulate the immune response, e.g., adjuvants, may be included in the compositions, e.g., in vaccines. Examples of chemical compounds used as adjuvants include, but are not limited to, aluminum compounds (e.g., alum, Alhydrogel), oils, block polymers, immune stimulating complexes, vitamins and minerals (e.g., vitamin E, vitamin A, selenium, and vitamin B12), Quil A (saponins), bacterial and fungal cell wall components (e.g., lipopolysaccarides, lipoproteins, and glycoproteins), hormones, cytokines, and co-stimulatory factors.

### Methods of Treatment

The invention features methods of vaccinating a mammal against candidiasis including administering to the animal a vaccine as described herein, thereby vaccinating the mammal against candidiasis. Additionally, the invention features methods of passive immunization of a mammal against candidiasis including administering to the mammal an effective amount of a pharmaceutical composition as described herein, thereby passively immunizing the mammal against candidiasis. Candidiasis may include, e.g., disseminated candidiasis, e.g., hematogenously disseminated candidiasis, or mucosal candidiasis. In some instances, the candidiasis is caused, e.g., by Candida albicans, Candida glabrata, Candida krusei, Candida parapsilosis, or Candida tropicalis. Other Candida species include Candida lusitaniae and Candida stellatoidea.

Additionally, the compositions and methods described herein may be used, e.g., to vaccinate a human at risk for the development of a S. aureus systemic infection or a skin or soft tissue infection against S. aureus. First, a human at risk for the development of a S. aureus infection or a S. aureus SSSI is identified. Second, the human is administered an immunogenic amount of a vaccine comprising a polypeptide described herein, in a pharmaceutically acceptable medium with or without an adjuvant. For example, the human is administered between one and three doses of Peptide 1 containing between 3 and 1000 μg of the peptide (SEQ ID NO:3) per dose, with multiple doses occurring at intervals of two weeks to six months.

It is expected that, following administration of the vaccine, the human is at decreased risk for the development of a S. aureus infection or an S. aureus SSSI for a period lasting from one month to several years or more.

Likewise, a human who is identified as having an S. aureus infection or an S. aureus SSSI may be treated by administration of an immunogenic amount of a pharmaceutical composition comprising a Peptide 1 in a pharmaceutically acceptable medium with or without an adjuvant. For example, the human is administered between one and three doses of Peptide 1 containing between 3 and 1000 μg of the peptide (SEQ ID NO:3) per dose, with multiple doses occurring at intervals of two weeks to six months.

Again, it is expected that, following administration of the pharmaceutical composition, the S. aureus SSSI of the human is decreased in severity.

The compositions and methods described herein may be used, e.g., to vaccinate a bovine species at risk for the development of a systemic S. aureus infection or even S. aureus skin or soft tissue infection against Staphylococcus aureus. In particular, the bovine species may be at risk of developing bovine mastitis caused by S. aureus. First, a bovine species at risk for the development of an S. aureus SSSI, e.g., bovine mastitis, is identified. For example, any milk-producing bovine may be considered to be at risk of developing bovine mastitis caused by S. aureus. Second, the bovine species is administered an immunogenic amount of a vaccine comprising one or more of the peptides disclosed herein in a pharmaceutically acceptable medium with or without an adjuvant. For example, the bovine species is administered between one and three doses of NDV-3 containing between 3 and 1000 μg of Peptide 1 (SEQ ID NO:3) per dose, with multiple doses occurring at intervals of two weeks to six months.

It is expected that, following administration of the vaccine, the bovine species is at decreased risk for the development of an S. aureus SSSI, e.g., bovine mastitis.

Likewise, a bovine species identified as having an S. aureus SSSI, e.g., bovine mastitis, may be treated by administration of an immunogenic amount of a pharmaceutical composition comprising one or more peptides disclosed herein in a pharmaceutically acceptable medium with or without an adjuvant. For example, the bovine species is administered between one and three doses of Peptide 1 (SEQ ID NO:3) containing between 3 and 1000 μg of the peptide (SEQ ID NO:3) per dose, with multiple doses occurring at intervals of two weeks to six months.

It is expected that, following administration of the pharmaceutical composition, the S. aureus SSSI, e.g., bovine mastitis, of the bovine species is decreased in severity.

Vaccines and antibody-containing pharmaceutical compositions (collectively “compositions”) as described herein can be administered prophylactically or therapeutically on their own or in combination with other art-known compositions that induce protective responses against pathogens (e.g., viral, bacterial, fungal, or parasitic pathogens), tumors or cancers, allergens, autoimmune disorders, or graft rejection. For example, the compositions can be administered simultaneously, separately, or sequentially, e.g., with another immunization vaccine, such as a vaccine for, e.g., influenza, malaria, tuberculosis, smallpox, measles, rubella, mumps, or any other vaccines known in the art.

Compositions as described herein can be delivered to a mammalian subject (e.g., a human or other mammal described herein) using a variety of known routes and techniques. For example, a composition can be provided as an injectable solution, suspension, or emulsion, and administered via intramuscular, subcutaneous, intradermal, intracavity, parenteral, epidermal, intraarterial, intraperitoneal, or intravenous injection using a conventional needle and syringe, or using a liquid jet injection system. Compositions can also be administered topically to skin or mucosal tissue, such as nasally, intratracheally, intestinal, rectally or vaginally, or provided as a finely divided spray suitable for respiratory or pulmonary administration. Other modes of administration include oral administration, suppositories, and active or passive transdermal delivery techniques.

The compositions described herein can be administered to a mammalian subject (e.g., a human or other mammal described herein) in an amount that is compatible with the dosage formulation and that will be prophylactically and/or therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one embodiment, a single dose is administered on a single occasion. In an alternative embodiment, a number of doses are administered to a subject on the same occasion but, for example, at different sites. In a further embodiment, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

In one embodiment, different compositions of the invention may be administered at different sites or on different occasions as part of the same treatment regime.

Different administrations may be performed on the same occasion, on the same day, one, two, three, four, five or six days apart, or one, two, three, four or more weeks apart. In some instances, administrations are 1 to 5 weeks apart, e.g., 2 to 4 weeks apart, such as 2 weeks, 3 weeks or 4 weeks apart. The schedule and timing of such multiple administrations can be optimised for a particular vaccine or pharmaceutical composition by one of skill in the art by routine trials.

Dosages

An adequate dose of the vaccines or antibody-containing pharmaceutical compositions described herein may vary depending on such factors as preparation method, administration method, age, body weight and sex of the patient, severity of symptoms, administration time, administration route, rate of excretion, and responsivity. A physician of ordinary skill in the art will easily determine and diagnose the administration dose effective for treatment.

Compositions may be prepared into unit-dose or multiple-dose preparations by those skilled in the art using a pharmaceutically acceptable carrier and/or excipient according to a method known in the art.

### Assessment

The following examples are intended to illustrate the invention. These are not meant to limit the invention in any way.

### Methods and Materials for Evaluating Treatment of Candidiasis

Candida Strains and Growth Conditions

C. albicans 15663, C. glabrata 31028, C. parapsilosis 22019 and C. tropicalis 4243 are clinical bloodstream isolates collected from Harbor-UCLA Medical Center. C. krusei 91-1159 was generously provided by Michael Rinaldi, San Antonio, Tex. C. albicans strains CAAH-31 and THE31 are as described in the literature. All tested strains were routinely grown in YPD (2% Bacto Peptone, 1% yeast extract, 2% dextrose). Cell densities were determined by counting in a hemacytometer.

Synthetic Peptides and Rabbit Polyclonal Antibodies

Peptides shown in Table 1 are synthesized using standard methods. To generate antibodies, the peptides may be purified and conjugated to keyhole limpet hemocyanin (KLH) before raising rabbit antiserum individually using a standard immunization protocol. Total IgG from pooled serum is affinity purified using Pierce Protein A plus Agarose (Thermo Scientific, Rockford, Ill.) prior to administering in passive immunization studies.

Immunization Protocol and Animal Studies

All active vaccinations are conducted according to standard methods. In brief, juvenile (10-12 week) Balb/C mice are vaccinated subcutaneously with 30 μg of peptide mixed with alum (2% Alhydrogel; Brenntag Biosector, Frederikssund, Denmark) as an adjuvant in phosphate buffered saline (PBS) on day 0, boosted with the same dose on day 21, then infected via the tail vein on day 35. Control mice are vaccinated with alum alone.

To test the efficacy of the vaccine in immunocompromised mice, mice are vaccinated as above prior to inducing neutropenia by intraperitoneal injection of 200 mg/kg of cyclophosphamide on day −2 followed by another dose of 100 mg/kg on day +7 relative to infection. This regimen results in approximately 10 days of leucopenia with reduction in neutrophil, lymphocyte and monocyte counts according to standard methods. For both immunocompetent and neutropenic mice differences in survival between vaccinated and adjuvant vaccinated mice are compared by the Log Rank test.

For passive immunization, immune IgG is administered intraperitoneally to naïve mice 2 h before infecting i.v. with C. albicans. Control mice are given isotype matching IgG (Innovative Research, USA). IgG doses are repeated 3 days after infection, and survival of mice was monitored twice daily.

Quantitative culturing of kidneys from vaccinated or control mice to be infected with different species of Candida is performed according to standard methods. In brief, mice are infected through tail veins. Kidneys are harvested 3 day post infection, homogenized, serially diluted in 0.85% saline, and quantitatively cultured on YPD that contained 50 μg/mL chloramphenicol. Colonies are counted after incubation of the plates at 37° C. for 24 to 48 h, and results are expressed as log CFU per gram of infected organ.

Concomitant with the fungal burden experiment, kidneys are removed aseptically from two mice per group for histopathological examination. Kidneys are immersed in zinc formalin fixative until examination. Fixed organs are dehydrated in graded alcohol solutions, embedded in paraffin, and cut into 6-μm-thick sections. Mounted sections are stained with Gomori methenamine silver and examined by light microscopy (Davis et al. (2000) Infect Immun 68: 5953-5959).

Enzyme-Linked Immunosorbent Assay (ELISA)

To test if a peptide in Table 1 induces an immune response, antibody titers of serum samples are collected from vaccinated and control mice are determined by ELISA in 96-well plates as previously described (Ibrahim et al. (2005) Infect Immun 73: 999-1005). Wells are coated at 100 μl per well with a peptide (e.g., one of more of peptide 2-11) at 5 μg/ml in PBS. Mouse sera are incubated for 1 h at room temperature following a blocking step with Tris-buffered saline (TBS; 0.01 M Tris HCl [pH 7.4], 0.15 M NaCl) containing 3% bovine serum albumin. The wells are then washed three times with TBS containing 0.05% Tween 20, followed by another three washes with TBS. Goat anti-mouse secondary antibody conjugated with horseradish peroxidase (Sigma) is added at a final dilution of 1:5000, and the plate is further incubated for 1 h at room temperature. Wells are then washed with TBS and incubated with substrate containing 0.1 M citrate buffer (pH 5.0), 50 mg of o-phenylenediamine (Sigma), and 10 μl of 30% H₂O₂. The color is allowed to develop for 30 min, after which the reaction is terminated by addition of 10% H₂SO₄ and the optical density (OD) at 490 nm is determined in a microtiter plate reader. Negative control wells received only diluent, and background absorbance is subtracted from the test wells to obtain final OD readings. The ELISA titer is taken as the reciprocal of the last serum dilution that gave a positive OD reading (i.e., more than the mean OD of negative control samples plus 2 standard deviations).

F(ab′)₂ Blocking Assay

To study the mechanism of protection mediated by anti-peptide (e.g., one described in Table 1) antibodies in phagocyte-mediated killing of C. albicans, HL-60 cells that have been differentiated to neutrophil-like phenotype are employed (Luo et al., (2010) J Infect Dis 201: 1718-1728). A killing assay is conducted in the presence of anti-peptide IgG or F(ab′)₂ fragments as described before (Luo (2010) J Infect Dis 201: 1718-1728). In brief, HL-60 cells are induced with 2.5 μM of retinoic acid and 1.3% DMSO for three days at 37° C. with 5% CO₂. Immune anti-Als3 or anti-CNA peptides (Table 1) sera are pooled and total IgG is isolated using protein A agarose (Thermo Scientific). Serum collected from the same rabbits prior to immunization with the peptides serves as control serum. The F(ab′)₂ fragments from immune or control IgG is purified with Pierce F(ab′)₂ Preparation Kit according to the manufacturer's instruction. SDS-PAGE analysis is utilized to indicate >95% of Fc fragment is digested. Next, C. albicans cells overexpressing or suppressing Als3 is incubated with 50 μg/ml of vaccinated or control F(ab′)₂ fragments on ice for 45 min. C. albicans cocultured with the F(ab′)₂ fragments is incubated with HL-60 derived neutrophils for 1 h at 37° C. with 5% CO₂ prior to sonication and quantitative culturing on YPD plates. % killing is calculated by dividing the number of CFU after coculturing with HL-60 derived neutrophils by the number of CFU from C. albicans incubated with media without neutrophil-like cells.

Statistical Analysis

The nonparametric log rank test is used to determine differences in the survival times of the mice. Neutrophil killing assay, titers of antibody, and tissue fungal burden is compared by the Mann-Whitney U test for unpaired comparisons. Correlations are calculated with the Spearman rank sum test. P values of <0.05 are considered significant.

Expected Results

Peptides that significantly improved survival and decreased fungal burden in immunocompetent mice challenged i.v. with C. albicans are taken as being useful in the invention. Similarly, peptides that statistically protect immunocompromised mice against candidiasis are useful in the invention. Mice protected from fungal infection after receiving purified IgG targeting a peptide disclosed herein in a dose specific manner are not only taken as an indication of the usefulness of passive immunization strategies for treating candidiasis but also for the usefulness of the peptide antigen used to raise an immune response. Peptide vaccines that substantially reduce tissue fungal burden in BALB/c mice challenged with several non-albicans species of Candida are likewise taken as being useful in the invention.

### Methods and Materials for Evaluating Treatment of a Staphylococcal Infection

Briefly, to determine whether a peptide (for example one or more of Peptides 3-11 as described herein) protects against S. aureus, female Balb/c mice are vaccinated with complete Freund's Adjuvant according to standard methods with a regimen on day 0, followed by a booster dose in Incomplete Freund's Adjuvant at 3 weeks. Two weeks following vaccination, mice are infected via the tail-vein with a lethal dose of S. aureus strain 67-0, which is methicillin-resistant and is known to be virulent in animal models. Peptides mediating improved long-term survival in these infected mice are taken as being useful in the invention.

Peptides may also be tested in the following murine model of skin or soft tissue infection. Peptide vaccination is evaluated across a dose range using a regimen of alhydrogel adjuvant. Doses of 3, 10, 30, 100, or 300 μg (IM) are studied in parallel. Primary vaccination (day 0) is followed by an identical boost on study day 21. Mice are infected with S. aureus 14 days after boost (study day 35). A subcutaneous skin/soft tissue abscess model is modified from Ding et al. (J. Bacteriol. 2008 190:7123-9) and/or Voyich et al. (J. Infect. Dis. 2006 194:1761-1770) for these studies. On study day 35, mice are anesthesized, flanks were shaved and sterilized, and 2×107 CFU inocula (without beads or matrix) is introduced into the subcutaneous compartment by injection (100 μl). A minimum of 20 mice per control or vaccine-regimen groups is used in each study. Abscess area/volume is then measured in each mouse flank during the study period up to 14 days post-challenge. To do so, mice are anesthetized, and the lesion site length (l) and width (w) is assessed to quantify abscess or dermonecrosis area (cm2). Abscess volume (cm³) is calculated per the formula for a spherical ellipsoid: [v=(π/6)×l×w2]. For quantitative culture analyses, at pre-selected times post-infection, mice were humanely sacrificed and processed for quantitative culture of abscesses. Each flank are aseptically dissected, the abscess removed and prepared for culture. Abscesses are individually homogenized, and serially diluted in sterile PBS for quantitative culture onto sheep blood agar plates. Cultures are incubated (37° C.) for 24 hours, and resulting colonies enumerated. For statistical analyses, differences in experimental results are compared based on power estimates indicating that 16-20 mice per group yields >85% power to detect 1 log difference in CFU per gram tissue, or 2 mm abscess area (a=0.05; Mann-Whitney U test. P values are defined according to standard methods.

Expected Results

Peptide vaccines that significantly reduce the abscess area, volume, or CFU densities in the murine model of MRSA skin or soft tissues assay are taken as being useful in the invention. Such results are taken to indicate that the peptide vaccine tested is useful as a means to prevent or mitigate MRSA skin infection or abscesses or both in mammals

### Additional Assessment Utilizing Human PBMCs

Useful peptide antigens (Table 1) are identified using human PBMCs obtained from individuals vaccinated using an Als3 or CNA polypeptide. The PBMCs are collected at various time points following vaccination and stored at −80° C. The PBMCs are thawed prior to use.

Once thawed, multiple PBMC samples are combined to prepare three pools of PBMCs: a Th1-responding PBMC pool, a Th17-responding PBMC pool, and a pool that includes Th1-responding PBMCs and Th17-responding PBMCs. Pooled PBMCs from individuals that were not vaccinated using an Als3 or CNA polypeptide (i.e., “unvaccinated subjects”) may also be used as a control or other controls as known by one skilled in the art.

In one working example, for an assay, ELISpot plates coated with antibodies to one or more specific human cytokines or chemokines, e.g., IFN-γ, lymphotoxin, IL-17A, IL-4, IL-5, IL-10, IL-13, or GRO may be used. Each pool of PBMCs are then activated in culture for 48 h and are distributed in 96-well ELISpot plates at 200,000 cells per well (inactivated PBMCs may be used as a control). Specific peptides and/or combinations of peptides are added to triplicate wells and incubated for 48-96 h and then the supernatants from each well are removed for analysis. The ELISpot plates are developed to reveal the spot forming units per well reflecting the number or cells in the well that produce the compound of interest (e.g., one or more cytokines or chemokines).

The data are assessed to determine whether a peptide or combination of peptides of the invention polarizes the immune response towards a Th1 response (e.g., an increase in production of IFN-g and/or lymphotoxin), a Th17 response (e.g., an increase in production of IL-17A), or a Th2 response (e.g., an increase in one or more of IL-4, IL-5, IL-10, and/or IL-13). Those peptide or peptide combinations that polarize the immune response towards a Th17 response, a Th1 response, or a Th1/Th17 response, relative to unstimulated PBMCs or stimulated PBMCs from unvaccinated subjects, are taken as being useful in the invention.

### Other Embodiments

All publications, patents, and patent applications mentioned in the above specification are hereby incorporated by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims

What is claimed is:

1. An isolated polypeptide comprising the amino acid sequence of any one of SEQ ID NOs: 3-11, or a variant sequence thereof having up to three substitutions, deletions, or additions to said amino acid sequence of any one of SEQ ID NOs: 3-11, wherein said polypeptide does not comprise more than 20 contiguous amino acids of SEQ ID NO: 1 or SEQ ID NO: 2 or SEQ ID NO:17.

2. An isolated polypeptide comprising the amino acid sequence of Formula I [ZNZPVSSBSFSYT]ₙ, wherein B is an amino acid selected from the group consisting of D and E; Z is an amino acid selected from the group consisting of F, H, P, T, W, and Y; and n is an integer from 1 to 10; and wherein said polypeptide does not comprise more than 20 contiguous amino acids of SEQ ID NO: 1 or SEQ ID NO: 2 or SEQ ID NO: 17.

3. An isolated polypeptide comprising the amino acid sequence of Formula II [ZNUJVOOBUFOYT]ₙ, wherein B is an amino acid selected from the group consisting of D and E; J is an amino acid selected from the group consisting of A, I, L, M, V, F, H, P, T, W, and Y; O is an amino acid selected from the group consisting of G, K, N, R, Q, S, T, Y, D, and E; U is an amino acid selected from the group consisting of D, E, S, T, and Y; Z is an amino acid selected from the group consisting of F, H, P, T, W, and Y; and n is an integer from 1 to 10; and wherein said polypeptide does not comprise more than 20 contiguous amino acids of SEQ ID NO: 1 or SEQ ID NO: 2 or SEQ ID NO:17.

4. The polypeptide of claim 1, comprising the amino acid sequence of any one of SEQ ID NOs: 3-11.

5. The polypeptide of claim 1, 2, 3 or 4, wherein the amino acid sequence of said polypeptide consists of between 14 and 20 amino acids.

6. The polypeptide of any one of claims 1-5, wherein the N-terminal amino acid residue or C-terminal amino acid residue of said polypeptide is cysteine.

7. An isolated conjugate comprising the polypeptide of any one of claims 1-6 conjugated to a carrier.

8. The conjugate of claim 7, wherein said carrier is keyhole limpet hemocyanin (KLH), CRM197, or tetanus toxoid.

9. The conjugate of claim 7, wherein said carrier is a phage, a yeast, a virus, virosome, or a recombinant virus-like particle.

10. The conjugate of claim 7, wherein said conjugate is a recombinant fusion protein.

11. A vaccine comprising an immunogenic amount of the polypeptide of any one of claims 1-6 or the conjugate of any one of claims 7-10, and a pharmaceutically acceptable excipient.

12. The vaccine of claim 11, comprising a mixture of distinct polypeptides of any one of claims 1-6 or conjugates of any one of claims 7-10.

13. The vaccine of claim 11 or 12, further comprising an adjuvant.

14. The vaccine of claim 13, wherein said adjuvant is Alhydrogel.

15. The vaccine of any one of claims 11-14, wherein said polypeptide or conjugate is produced synthetically.

16. The vaccine of any one of claims 11-14, wherein said polypeptide or conjugate is produced recombinantly.

17. The vaccine of any one of claims 11-16 for use in the vaccination of a mammal against candidiasis.

18. The vaccine of claim 17, wherein said mammal is a human.

19. The vaccine of claim 17 or 18, wherein said vaccine is to be administered by intramuscular, subcutaneous, or intradermal administration.

20. The vaccine of claim 17 or 18, wherein said vaccine is to be administered by intramuscular administration.

21. The vaccine of any one of claims 17-20, wherein said vaccination further comprises administering a booster dose.

22. The vaccine of any one of claims 17-21, wherein said candidiasis is disseminated candidiasis.

23. The vaccine of claim 22, wherein said disseminated candidiasis is hematogenously disseminated candidiasis.

24. The vaccine of any one of claims 17-21, wherein said candidiasis is mucosal candidiasis.

25. The vaccine of any one of claims 17-24, wherein said candidiasis is caused by Candida albicans, Candida glabrata, Candida krusei, Candida parapsilosis, or Candida tropicalis.

26. A method of vaccinating a mammal against candidiasis or a staphylococcus infection or both comprising administering to said mammal the vaccine of any one of claims 11-16, thereby vaccinating said mammal against candidiasis or a staphylococcus infection or both.

27. The method of claim 26, wherein said mammal is a human.

28. The method of claim 26 or 27, wherein said vaccine is administered by intramuscular, subcutaneous, or intradermal administration.

29. The method of claim 26 or 27, wherein said vaccine is administered by intramuscular administration.

30. The method of any one of claims 26-29, wherein said administering further comprises administering a booster dose.

31. The method of any one of claims 26-30, wherein said candidiasis is disseminated candidiasis.

32. The method of claim 31, wherein said disseminated candidiasis is hematogenously disseminated candidiasis.

33. The method of any one of claims 26-30, wherein said candidiasis is mucosal candidiasis.

34. The method of any one of claims 26-33, wherein said candidiasis is caused by Candida albicans, Candida glabrata, Candida krusei, Candida parapsilosis, or Candida tropicalis.

35. The method of any one of claims 26-30, wherein said staphylococcus infection is a systemic infection or is a SSTI infection.

36. The method of any one of claims 26-30 and 35, wherein said staphylococcal infection is caused by MRSA.

37. A method of producing a chimeric vaccine comprising the steps of:
(a) providing a phage, yeast, or virus;
(b) inserting into said phage, yeast, or virus a nucleic acid molecule that encodes the polypeptide of any one of claims 1-6;
(c) allowing expression of said polypeptide in said phage, yeast, or virus;
(d) isolating said phage, yeast, or virus of step (c) comprising said expressed polypeptide; and
(e) adding a pharmaceutically acceptable excipient to said isolated phage, yeast, or virus of step (d).

38. The method of claim 35, wherein said polypeptide is displayed on the surface of said phage, yeast, or virus following step (c).

39. An isolated monoclonal antibody that binds to the polypeptide of any one of claims 1-6.

40. The antibody of claim 39, wherein said antibody is human or humanized.

41. The antibody of claim 39, wherein said antibody is chimeric.

42. The antibody of any one of claims 39-41, wherein said antibody is produced recombinantly.

43. A diagnostic composition comprising the antibody of any one of claims 39-42.

44. A pharmaceutical composition comprising the antibody of any one of claims 39-42 and a pharmaceutically acceptable excipient.

45. The pharmaceutical composition of claim 44, comprising a mixture of antibodies of any one of claims 39-42 with a plurality of distinct specificities.

46. A pharmaceutical composition comprising polyclonal antibodies that bind to the polypeptide of any one of claims 1-6, or that bind to a mixture of distinct polypeptides of any one of claims 1-6.

47. The pharmaceutical composition of any one of claims 44-46 for use in the passive immunization of a mammal against candidiasis or a staphylococcal infection.

48. The pharmaceutical composition of claim 47, wherein said mammal is a human.

49. The pharmaceutical composition of claim 47 or 48, wherein said pharmaceutical composition is administered by intramuscular, subcutaneous, or intradermal administration.

50. The pharmaceutical composition of claim 47 or 48, wherein said pharmaceutical composition is administered by intramuscular administration.

51. The pharmaceutical composition of any one of claims 47-50, wherein said candidiasis is disseminated candidiasis.

52. The pharmaceutical composition of claim 51, wherein said disseminated candidiasis is hematogenously disseminated candidiasis.

53. The pharmaceutical composition of any one of claims 47-50, wherein said candidiasis is mucosal candidiasis.

54. The pharmaceutical composition of any one of claims 47-53, wherein said candidiasis is caused by Candida albicans, Candida glabrata, Candida krusei, Candida parapsilosis, or Candida tropicalis.

55. The pharmaceutical composition of any one of claims 47-50, wherein said staphylococcus infection is a systemic infection or is a SSTI infection.

56. The pharmaceutical composition of any one of claims 47-50 and 55, wherein said staphylococcal infection is caused by MRSA.

57. A method of passive immunization of a mammal against candidiasis comprising administering to said mammal an effective amount of the pharmaceutical composition of any one of claims 44-46, thereby passively immunizing said mammal against said candidiasis.

58. The method of claim 57, wherein said mammal is a human.

59. The method of claim 57 or 58, wherein said pharmaceutical composition is administered by intramuscular, subcutaneous, or intradermal administration.

60. The method of claim 57 or 58, wherein said pharmaceutical composition is administered by intramuscular administration.

61. The method of any one of claims 57-60, wherein said candidiasis is disseminated candidiasis.

62. The method of claim 61, wherein said disseminated candidiasis is hematogenously disseminated candidiasis.

63. The method of any one of claims 57-60, wherein said candidiasis is mucosal candidiasis.

64. The method of any one of claims 57-63, wherein said candidiasis is caused by Candida albicans, Candida glabrata, Candida krusei, Candida parapsilosis, or Candida tropicalis.

65. A method of passive immunization of a mammal against a staphylococcus infection comprising administering to said mammal an effective amount of the pharmaceutical composition of any one of claims 44-46, thereby passively immunizing said mammal against said staphylococcus infection.

66. The method of claim 65, wherein said mammal is a human.

67. The method of claim 65 or 66, wherein said pharmaceutical composition is administered by intramuscular, subcutaneous, or intradermal administration.

68. The method of claim 65 or 66 wherein said pharmaceutical composition is administered by intramuscular administration.

69. The method of any one of claims 65-68, wherein said staphylococcus infection is a systemic infection or is a SSTI infection.

70. The method of anyone of claims 65-69, wherein said staphylococcal infection is caused by MRSA.


SEQUENCE LISTING (18 sequences)

SEQ ID NO: 1 - protein, 1588 aa, Candida albicans
MLLQFLLLSLCVSVATAKVITGVFNSFDSLTWTRAGNYAYKGPNRPTWNAVLGWSLDGTSANPGDTFTLNMPCVFKFITDQTSVDLTADGVKYATCQFYSGEEFTTFSSLKCTVSNTLTSSIKALGTVTLPISFNVGGTGSLVDLESSKCFKAGTNTVTFNDGDKKISIDVDFEKTNEDASGYFIASRLIPSINKASITYVAPQCANGYTSGAMGFTIGSGDTTIDCSNVHVGITKGLNDWNFPVSSDSLSYNKTCSSTGISITYENVPAGYRPFFDVYTSVSDQNRQLKYTNDYACVGSSLQSKPFNLRLRGYNNSEANSNGFVIVATTRTVTDSTTAVTTLPFNPSIDKTKTIEILQPIPTTTITTSYVGVTTSYSTKTAPIGETATVIVDVPYHTTTTVTSEWTGTITTTTTRTNPTDSIDTVVVQVPSPNPTVSTTEYWSQSYATTTTITAPPGGTDTVIIREPPNHTVTTTEYWSQSYATTTTVTAPPGGTDSVIIREPPNPTVTTTEYWSQSYATTTTITAPPGGTDTVIIREPPNHTVTTTEYWSQSYATTTTVTAPPGGTDSVIIREPPNPTVTTTEYWSQSYTTTTTVTAPPGGTDSVIIREPPNPTVTTTEYWSQSYATTTTVTATPGGTDTVIIREPPNYTVTTTEYWSQSYATTTTVTAPPGGTDTVIIREPPNYTVTTTEYWSQSYATTTTVTAPPGGTDTVIIREPPNYTVTTTEYWSQSYATTSTITAPPGGTDTVIIREPPNYTVTTTEYWSQSYATTTTVTGPPGGTDTVIIREPPSPTVTTTEYWSQSYATTTTVTAPPGGTATVIIKEPPNYTVTTTEYWSQSYATTTTVTGPPGGTDTVIIREPPNPTVTTTEYWSQSFATTTTVTAPPGGTDSVIIREPPNPTVTTTEYWSQSYATTTTVTAPPGGTDSVIIREPPNPTVTTTEYWSQSYTTTTTVTAPPGGTDSVIIREPPNPTVTTTEYWSQSYATTTTVTATPGGTDTVIIREPPNYTVTTTEYWSQSYATTTTVTAPPGGTDTVIIREPPNYTVTTTEYWSQSYATTTTVTAPPGGTDTVIIREPPNHTVTTTEYWSQSYTTTTTVIAPPGGTDSVIIREPPNPTVTTTEYWSQSYATTTTITAPPGETDTVLIREPPNHTVTTTEYWSQSFATTTTVTAPPGGTDTVIIREPPNHTVTTTEYWSQSYATTTTITAPPGETDTVLIREPPNHTVTTTEYWSQSYATTTTIIAPPGETDTVLIREPPNPTVTTTEYWSQSYTTATTVTAPPGGTDTVIIYDTMSSSEISSFSRPHYTNHTTLWSTTWVIETKTITETSCEGDKGCSWVSVSTRIVTIPNNIETPMVTNTVDTTTTESTLQSPSGIFSESGVSVETESSTFTTAQTNPSVPTTESEVVFTTKGNNGNGPYESPSTNVKSSMDENSEFTTSTAASTSTDIENETIATTGSVEASSPIISSSADETTTVTTTAESTSVIEQQTNNNGGGNAPSATSTSTSSPSTTTTANNDSVITSTTSINQSQSQSNSDTQQTTLSQQMTSSLVSLHMLTTFDGSGSVIQHSTWLCGLITLLSLFI

SEQ ID NO: 2 - protein, 1183 aa, Staphylococcus aureus
MNKNVLKFMVFIMLLNIITPLFNKNEAFAARDISSTNVTDLTVSPSKIEDGGKTTVKMTFDDKNGKIQNGDMIKVAWPTSGTVKIEGYSKTVPLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKSYVSKDITIKDQIQGGQQLDLSTLNINVTGTHSNYYSGQSAITDFEKAFPGSKITVDNTKNTIDVTIPQGYGSYNSFSINYKTKITNEQQKEFVNNSQAWYQEHGKEEVNGKSFNHTVHNINANAGIEGTVKGELKVLKQDKDTKAPIANVKFKLSKKDGSVVKDNQKEIEIITDANGIANIKALPSGDYILKEIEAPRPYTFDKDKEYPFTMKDTDNQGYFTTIENAKAIEKTKDVSAQKVWEGTQKVKPTIYFKLYKQDDNQNTTPVDKAEIKKLEDGTTKVTWSNLPENDKNGKAIKYLVKEVNAQGEDTTPEGYTKKENGLVVTNTEKPIETTSISGEKVWDDKDNQDGKRPEKVSVNLLANGEKVKTLDVTSETNWKYEFKDLPKYDEGKKIEYTVTEDHVKDYTTDINGTTITNKYTPGETSATVTKNWDDNNNQDGKRPTEIKVELYQDGKATGKTAILNESNNWTHTWTGLDEKAKGQQVKYTVEELTKVKGYTTHVDNNDMGNLIVTNKYTPETTSISGEKVWDDKDNQDGKRPEKVSVNLLADGEKVKTLDVTSETNWKYEFKDLPKYDEGKKIEYTVTEDHVKDYTTDINGTTITNKYTPGETSATVTKNWDDNNNQDGKRPTEIKVELYQDGKATGKTAILNESNNWTHTWTGLDEKAKGQQVKYTVEELTKVKGYTTHVDNNDMGNLIVTNKYTPETTSISGEKVWDDKDNQDGKRPEKVSVNLLANGEKVKTLDVTSETNWKYEFKDLPKYDEGKKIEYTVTEDHVKDYTTDINGTTITNKYTPGETSATVTKNWDDNNNQDGKRPTEIKVELYQDGKATGKTAILNESNNWTHTWTGLDEKAKGQQVKYTVDELTKVNGYTTHVDNNDMGNLIVTNKYTPKKPNKPIYPEKPKDKTPPTKPDHSNKVKPTPPDKPSKVDKDDQPKDNKTKPENPLKELPKTGMKIITSWITWVFIGILGLYLILRKRFNS

SEQ ID NO: 3 - protein, 9 aa, Candida albicans
PVSSDSLSY

SEQ ID NO: 4 - protein, 9 aa, Candida albicans
SSDSLSYNK

SEQ ID NO: 5 - protein, 9 aa, Candida albicans
VTDSTTAVT

SEQ ID NO: 6 - protein, 9 aa, Candida albicans
VTSEWTGTI

SEQ ID NO: 7 - protein, 9 aa, Candida albicans
PTDSIDTVV

SEQ ID NO: 8 - protein, 9 aa, Staphylococcus aureus
STNVTDLTV

SEQ ID NO: 9 - protein, 9 aa, Staphylococcus aureus
VTSETNWKY

SEQ ID NO: 10 - protein, 9 aa, Staphylococcus aureus
VTEDHVKDY

SEQ ID NO: 11 - protein, 9 aa, Staphylococcus aureus
PTEIKVELY

SEQ ID NO: 12 - protein, 130 aa, Artificial Sequence
  Description of Artificial Sequence Synthetic polypeptide
XNXPVSSXSFSYTXNXPVSSXSFSYTXNXPVSSXSFSYTXNXPVSSXSFSYTXNXPVSSXSFSYTXNXPVSSXSFSYTXNXPVSSXSFSYTXNXPVSSXSFSYTXNXPVSSXSFSYTXNXPVSSXSFSYT

SEQ ID NO: 13 - protein, 18 aa, Artificial Sequence
  Description of Artificial Sequence Synthetic peptide
KGLNDWNYPVSSESFSYT

SEQ ID NO: 14 - protein, 17 aa, Artificial Sequence
  Description of Artificial Sequence Synthetic peptide
KGLNDWNYPVSSESFSY

SEQ ID NO: 15 - protein, 16 aa, Artificial Sequence
  Description of Artificial Sequence Synthetic peptide
KGLNDWNYPVSSESFS

SEQ ID NO: 16 - protein, 9 aa, Artificial Sequence
  Description of Artificial Sequence Synthetic peptide
WNYPVSSES

SEQ ID NO: 17 - protein, 989 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQNTDASNKDVVSQAVNPSTPRMRAFSLAAVAADAPAAGTDITNQLTDVKVTIDSGTTVYPHQAGYVKLNYGFSVPNSAVKGDTFKITVPKELNLNGVTSTAKVPPIMAGDQVLANGVIDSDGNVIYTFTDYVDNKENVTANITMPAYIDPENVTKTGNVTLTTGIGTNTASKTVLIDYEKYGQFHNLSIKGTIDQIDKTNNTYRQTIYVNPSGDNVVLPALTGNLIPNTKSNALIDAKNTDIKVYRVDNANDLSESYYVNPSDFEDVTNQVRISFPNANQYKVEFPTDDDQITTPYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSASDSDSASDSDSASDSDSASDSDSASDSDSASDSDSASDSDSASDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSESDSDSDSDSDSDSDSDSDSDSDSESDSDSDSDSDSESDSDSDSDSDSDSASDSDSGSDSDSSSDSDSDSTSDTGSDNDSDSDSNSDSESGSNNNVVPPNSPKNGTNASNKNEAKDSKEPLPDTGSEDEANTSLIWGLLASLGSLLLFRRKKENKDKK

SEQ ID NO: 18 - protein, 130 aa, Artificial Sequence
  Description of Artificial Sequence Synthetic polypeptide
XNXXVXXXXFXYTXNXXVXXXXFXYTXNXXVXXXXFXYTXNXXVXXXXFXYTXNXXVXXXXFXYTXNXXVXXXXFXYTXNXXVXXXXFXYTXNXXVXXXXFXYTXNXXVXXXXFXYTXNXXVXXXXFXYT